(19) United States
(12) Patent Application Publication
(10) Pub. No.: US 2017/0231893 A1
(43) Pub. Date: Aug. 17, 2017

(54) AGENTS FOR THE TEMPORARY SHAPING OF KERATIN-CONTAINING FIBERS, INCLUDING A HYDROPHOBICALLY MODIFIED METAL OXIDE POWDER, A FILM-FORMING POLYMER AND ETHANOL

(71) Applicant: Henkel AG & Co. KGaA, Duesseldorf (DE)

(72) Inventors: Thorsten Knappe, Schenefeld (DE); Ulrike Heinsohn, Hamburg (DE)

(73) Assignee: Henkel AG & Co. KGaA, Duesseldorf (DE)

(21) Appl. No.: 15/586,492

(22) Filed: May 4, 2017

Related U.S. Application Data
(63) Continuation of application No. PCT/EP2015/072336, filed on Sep. 29, 2015.

(30) Foreign Application Priority Data
Nov. 7, 2014 (DE) 102014222802.6

(51) Int. Cl. A61K 8/81; A61K 8/25; B65D 83/14; A45D 7/04; A45D 34/04; A61Q 5/06; A61K 8/34

(52) U.S. Cl. CPC A61K 8/8158; A61Q 5/06; A61K 8/25; A61K 8/34; A45D 7/04; A45D 34/04; B65D 83/752; A61K 2800/87

(57) ABSTRACT

Cosmetic agents for temporarily shaping keratin fibers, comprising a) a cosmetic preparation, comprising, based on the total weight of the cosmetic preparation, a1) 0.05 to 5.0 wt.-% of at least one hydrophobically modified metal oxide powder; a2) 0.1 to 10 wt.-% of at least one film-forming polymer; and a3) 40 to 95 wt.-% of ethanol, are characterized by a high level of hold and volume.

### FIELD OF THE INVENTION

The present invention generally relates the technical field of the temporary reshaping of keratin-containing fibers, in particular human hair. The subject of the invention concerns cosmetic agents, including at least one hydrophobized metal oxide powder, a film-forming polymer, and ethanol, as well as cosmetic products, comprising a dispensing device with a spray valve and the aforementioned cosmetic agents. Moreover, the subject of the present invention is the use of these cosmetic agents and products for the temporary reshaping of keratin-containing fibers and suitable invention methods.

### BACKGROUND OF THE INVENTION

Today, an attractive hairstyle is generally regarded as an essential part of a well-groomed appearance. In this regard, based on current fashion trends, hairstyles time and again are considered chic that for many hair types can only be formed or sustained over a longer period up to several days by the use of active setting substances. Hair treatment agents used for a permanent or temporary shaping of hair therefore play an important role. Whereas during permanent reshaping the chemical structure of the keratin-containing fibers is modified by reduction and oxidation, such modifications of the chemical structure do not occur in temporary reshaping. Suitable agents for temporary shaping typically include synthetic polymers and/or waxes as an active setting substance.

The most important property of an agent for the temporary shaping of keratinic fibers, also called a styling agent hereinafter, is to give the treated fibers the strongest possible hold in the new modelled shape, i.e., a shape imposed on the fibers. If the keratinic fibers are human hair, then one also speaks of a strong hairstyle hold or of a high degree of hold of the styling agent. The hairstyle hold is determined substantially by the type and amount of the employed active setting substances, but the other components of the styling agent and the invention form may also have an effect.

In addition to a high degree of hold, styling agents should satisfy a wide range of further requirements. These can be divided roughly into properties on the hair, properties of the particular formulation, e.g., of the sprayed aerosol or non-aerosol, and properties related to the handling of the styling agent, particular importance being attached to the properties on the hair. Mention can be made in particular of humidity resistance, low tackiness, and a balanced conditioning effect. Furthermore, a styling agent should be universally usable, if possible, for all hair types and be gentle to the hair and skin.

In order to satisfy the various requirements, many synthetic polymers for use in styling agents were already developed in the prior art as active setting substances. The polymers can be divided into cationic, anionic, nonionic, and amphoteric setting polymers. The polymers when used on hair ideally form a polymer film that, on the one hand, gives the hairstyle a strong hold but, on the other, is sufficiently flexible not to break under stress. If the polymer film is too brittle, so-called film plaques or residues form that detach as the hair moves and give the impression that the user of the particular styling agent has dandruff. Similar problems arise if waxes are used as the active setting substance in the styling agent.

Agents for supporting the temporary reshaping of keratin-containing fibers can be produced, for example, as a hairspray, hair wax, hair gel, or hair foam. Application by means of a spray device in the form of a spray in particular is enjoying a high popularity. However, application by means of a spray device requires additional measures depending on nature of the styling agent. Thus, for example, the spray application of thickened hair gels is possible so far in the prior art only with the use of a certain combination of thickener and styling polymer, because sufficient atomization of the hair gel by means of the spray application can only be assured in this way. The combination, used in the prior art, of a carbomer and PVP/VA styling polymers can in fact be sufficiently atomized, but does not have a satisfactory long-lasting hold and volume effect and therefore no longer meets the needs of the consumer.

The object of the present invention, therefore, was to provide agents for the temporary shaping of keratin-containing fibers, which can be easily applied as a targeted spray mist to keratin-containing fibers. Furthermore, the agents for the temporary shaping of keratin-containing fibers should have a high degree of hold, in particular a long-lasting degree of hold, and a high volume effect.

### BRIEF SUMMARY OF THE INVENTION

A first subject of the present invention, therefore, is a cosmetic agent for the temporary shaping of keratinic fibers, comprising

a) a cosmetic preparation including, based on the total weight of the cosmetic preparation,
a1) 0.05 to 5.0% by weight of at least one hydrophobically modified metal oxide powder,
a2) 0.1 to 10% by weight of at least one film-forming polymer,
a3) 40 to 95% by weight of ethanol.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

It was now found surprisingly that an ethanolic preparation of a special combination of hydrophobically modified metal oxide powder with a film-forming polymer is suitable for achieving the above-described object. This specific combination is characterized not only by a high long-lasting hold and a good volume effect, but moreover can be nebulized outstandingly and is therefore also suitable for spray application. The use of a powdered composition based on hydrophobized silicon dioxide powder for the temporary shaping of keratinic fibers is described in the international patent application WO 2007051511 A1.

The agents of the invention include a hydrophobically modified metal oxide powder as the first essential component. Agents preferred in regard to their cosmetic action, producibility, and appliability are characterized in that the weight proportion of the hydrophobically modified metal oxide powder a1) in terms of the total weight of cosmetic preparation a) is 0.1 to 4.0% by weight, preferably 0.15 to 3.0% by weight, and in particular 0.2 to 2.0% by weight.

Metal oxides of this kind that have been modified at least on the surface of the particles such that the modified particle is wetted less by water than the unmodified particle are to be understood as “hydrophobically modified” or “hydrophobized” in the context of the invention. Silanized, hydrophobized metal oxides are particularly preferred. At least one representative of the group formed by silanes, halosilanes, alkoxysilanes, and silazanes is preferably suitable according to the invention as a reagent for silanizing the metal oxide. Preferably suitable hydrophobized metal oxides of the hydrophobized metal oxide powder are selected according to the invention from at least one representative of the group formed by hydrophobized silicates, hydrophobized aluminosilicates, hydrophobized titanium dioxide, and hydrophobized silicon dioxide. Hydrophobized silicates have proven to be particularly suitable for producing the cosmetic agents of the invention, wherein pyrogenic silicic acid aftertreated by silanization or by reaction with polydimethylsiloxane has particular advantages.

The agents of the invention preferably include hydrophobized silicon dioxide. The type of hydrophobically modified silicon dioxide a1) is limited in principle, but because of the cosmetic properties of suitable agents preferably at least one silanized, hydrophobized silicon dioxide is used as the hydrophobized metal oxide powder.

Preferably, at least one representative of the group formed by silanes, halosilanes, alkoxysilanes, and silazanes is suitable according to the invention as a reagent for silanizing the silicon dioxide.

Preferred representatives of the group of silanes are hexa(C₁-C₂₀) alkyl disilanes, particularly hexamethyldisilane.

If a halosilane is used as the silylating agent, selected as the preferred halosilane is at least one compound from the group formed by the compounds

[(C₁-C₂₀)alkyl]_{z′}SiX_{(4-z′)}

X₃Si[(CH₂)ₙ—R]

X₂[(C₁-C₂₀)alkyl]Si(CH2)ₙ—R

[(C₁-C₂₀)alkyl]_{(y′+1)}[R—(CH₂)ₙ]_{(2-y′)}SiX

where
X denotes a chlorine, bromine, or iodine atom,
z′ is a number 1, 2, or 3,
y′ is a number 0, 1, or 2,
n is an integer from 1 to 20, and
R stands for a group from
(C₁-C₁₀) alkyl-, aryl-, (C₁-C₆) perfluoroalkyl-, —NH₂, —N₃, —SCN, —CH═CH₂, —O(O)C—C(CH₃)═CH₂, —OCH₂—CH═CH₂, —NHC(O)—N—C(O)—(CH₂)₅

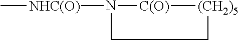

—NH—C(O)O-Me, —NH—C(O)O-Et, —NH—(CH₂)₃—Si(O(C₁-C₆)alkyl)₃.

If an alkoxysilane is used as the silylating agent, selected as the preferred alkoxysilane is at least one compound from the group formed by the compounds

[(C₁-C₂₀)alkylO]_{z}Si(C₁-C₂₀)alkyl_{(4-z)}

[(C₁-C₂₀)alkylO]_{z}Si[(CH₂)ₙ—R]_{(4-z)}

[(C₁-C₂₀)alkylO]₂[(C₁-C₂₀)alkyl]Si(CH₂)ₙ—R

[(C₁-C₂₀)alkylO][(C₁-C₂₀)alkyl]₂Si(CH₂)ₙ—R

[(C₁-C₂₀)alkylO][(C₁-C₂₀)alkyl]Si[(CH₂)ₙ—R]₂

(C₁-C₂₀ alkyl)₃SiO—C(CH₃)═N—Si(C₁-C₂₀)alkyl₃,

where
n is an integer from 1 to 20, and
z denotes a number 1, 2, or 3,
R stands for a group from
(C₁-C₂₀) alkyl-, aryl-, (C₁-C₆) perfluoroalkyl-, —NH₂, —N₃, —SCN, —CH═CH₂, —O(O)C—C(CH₃)═CH₂, —OCH₂—CH═CH₂,

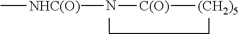

—NH—C(O)O-Me, —NH—C(O)O-Et, —NH—(CH₂)₃—Si(O(C₁-C₆)alkyl)₃.

Selected as the preferred silazane is at least one compound from the class of disilazanes, in particular at least one compound from disilazanes of the formula

R′₂R″Si—NH—SiR′₂R″,

where
R′ denotes a (C₁-C₂₀) alkyl group and
R″ denotes a (C₁-C₂₀) alkyl group or a vinyl group. A particularly preferred silazane is hexamethyldisilazane.

All of the aforesaid alkyl groups, whether (C₁-C₆) alkyl, (C₁-C₁₀) alkyl, or (C₁-C₂₀) alkyl, can be both cyclic and linear or branched. Examples of alkyl groups usable according to the invention are methyl, ethyl, n-propyl, isopropyl, n-butyl, cyclopentyl, cyclohexyl, n-decyl, lauryl, myristyl, cetyl, stearyl, isostearyl, and behenyl.

An example of an aryl group of the invention is the phenyl group.

Examples of a (C₁-C₆) perfluoroalkyl group of the invention are trifluoromethyl, perfluoroethyl, perfluoropropyl, and perfluorohexyl.

Used preferably are hydrophobized silicon dioxides obtained by silanization of pyrogenic silicon dioxide.

Silanized, hydrophobized silicon dioxides are selected particularly preferably from at least one compound of the group formed by trimethyl silylate-coated silicon dioxide, dimethyl silylate-coated silicon dioxide, and octyl silylate-coated silicon dioxide.

Preferred cosmetic agents are characterized in that the hydrophobically modified metal oxide powder a1) is selected from the group of hydrophobized silicates obtained by silanization of pyrogenic silicon dioxide.

A variety of suitable hydrophobically modified silicon dioxides is commercially available. Recited as examples are Aerosil® R104 V, Aerosil® R106, Aerosil® R202, Aerosil® R805, Aerosil® R812, Aerosil® R812S, Aerosil® R972, and Aerosil® R8200, all from Degussa, and HDK® H2000, HDK® H2050, and HDK® H3004, all from Wacker. It is very particularly preferred to use the silicon dioxide with the INCI name Silica Dimethyl Silylate, which is marketed by the company Degussa under the name Aerosil® R972. In summary, cosmetic agents are preferred in which the hydrophobically modified metal oxide powder a1) is selected from the compounds with the INCI names: Silica Dimethyl Silylate (e.g., Aerosil R792, Aerosil R794), Silica Dimethicone Silylate (e.g., Aerosil R202), and Silica Silylate (e.g., Aerosil R805, Aerosil R812, Aerosil R816). Compounds with the INCI name Silica Dimethyl Silylate are particularly preferred.

The hydrophobically modified silicon dioxides with the INCI name Silica Dimethyl Silylate enable, in comparison with the customary previously described hydrophobically modified silicon dioxides, a simplified production of cosmetic agents of the invention, which are notable, moreover, by an improved applicability and cosmetic effect. The particularly preferred hydrophobized silicon dioxides with the INCI name Silica Dimethyl Silylate can be produced, for example, by reacting pyrogenic silicic acid with dimethyldichlorosilane. A particularly preferred subject of the present application, therefore, is a cosmetic agent that includes a hydrophobically modified metal oxide powder, obtained by reacting pyrogenic silicic acid with dimethyldichlorosilane.

The particle diameter of the primary particles of preferred hydrophobically modified metal oxides a1), in particular the hydrophobically modified metal oxides with the INCI name Silica Dimethyl Silylate, is preferably less than 5 μm, particularly preferably less than 1 μm, and in particular between 1 and 50 nm.

Preferred, furthermore, are hydrophobically modified metal oxides a1) of this kind, in particular hydrophobically modified metal oxides with the INCI name Silica Dimethyl Silylate, which have a specific BET surface between 10 and 400 m²/g, preferably between 40 to 300 m²/g, and in particular 80 to 150 m²/g.

The powdered compositions of the invention include a film-forming polymer b) as the second essential component. It has emerged as advantageous for the use and the cosmetic effect of the cosmetic agents, if the weight proportion of film-forming polymer a2) in terms of the total weight of cosmetic preparation a) is 0.2 to 8.0% by weight, preferably 0.4 to 6.0% by weight, and in particular 0.5 to 5.0% by weight.

A film-forming polymer a2) particularly preferred because of its cosmetic effect is a copolymer obtained by polymerizing the monomers: N-tert-octylacrylamide, acrylic acid, and tert-butylaminoethyl methacrylic acid. Preferred film-forming polymers a2) consist of at least 90% by weight, preferably at least 95% by weight, and in particular at least 97% by weight of the monomers: N-tert-octylacrylamide, acrylic acid, and tert-butylaminoethyl methacrylic acid. Particularly preferred film-forming polymers a2) were obtained exclusively from the monomers: N-tert-octylacrylamide, acrylic acid, and tert-butylaminoethyl methacrylic acid. The above-described particularly preferred film-forming polymers a2) are marketed, for example, under the name Amphomer® (INCI name: Octylacrylamide/Acrylates/Butylaminoethyl Methacrylate Copolymer) from the company National Starch.

Preferred cosmetic agents are characterized in that film-forming polymer a2) is a copolymer of the monomers
i) N-tert-octylacrylamide,
ii) acrylic acid,
iii) tert-butylaminoethyl methacrylic acid,
iv) and optionally other monomers.

Preferred cosmetic agents include as film-forming polymer a2) at least 50% by weight, preferably at least 70% by weight, particularly preferably at least 90% by weight, and in particular at least 95% by weight of a copolymer obtained by polymerizing the monomers: N-tert-octylacrylamide, acrylic acid, and tert-butylaminoethyl methacrylic acid. Particularly preferred are cosmetic agents that include as film-forming polymer a2) solely a copolymer obtained by polymerizing the monomers: N-tert-octylacrylamide, acrylic acid, and tert-butylaminoethyl methacrylic acid.

The following have proven useful as further usable film-forming polymers a2) that can be used alternatively to the copolymers of N-tert-octylacrylamide, acrylic acid, and tert-butylaminoethyl methacrylic acid or in combination with these copolymers:
copolymer from the group of polyvinylpyrrolidone and vinylpyrrolidone/vinyl acetate copolymers, preferably polyvinylpyrrolidones
copolymer of the monomers: acrylic acid, ethyl acrylate, and tert-butylacrylamide.

Other preferred cosmetic agents are therefore characterized in that film-forming polymer a2) is
a copolymer from the group of polyvinylpyrrolidone and vinylpyrrolidone/vinyl acetate copolymers, preferably polyvinylpyrrolidones, or
a copolymer of the monomers
   i) acrylic acid,
   ii) ethyl acrylate,
      iii) tert-butylacrylamide.

Film-forming polymers a2) are used in the cosmetic agents preferably in partially neutralized or neutralized form. Preferably at least one alkanolamine is used for neutralization. The alkanolamines that can be used as alkalizing agents are preferably selected from primary amines having a C₂-C₆ alkyl parent substance, which carries at least one hydroxyl group. Particularly preferred alkanolamines are selected from the group, formed by 2-aminoethan-1-ol (monoethanolamine), tris(2-hydroxyethyl)amine (triethanolamine), 3-aminopropan-1-ol, 4-aminobutan-1-ol, 5-aminopentan-1-ol, 1-aminopropan-2-ol, 1-aminobutan-2-ol, 1-aminopentan-2-ol, 1-aminopentan-3-ol, 1-aminopentan-4-ol, 3-amino-2-methylpropan-1-ol, 1-amino-2-methylpropan-2-ol, 3-aminopropane-1,2-diol, and 2-amino-2-methylpropane-1,3-diol. Alkanolamines very particularly preferred according to the invention are selected from the group comprising 2-aminoethan-1-ol, 2-amino-2-methylpropan-1-ol, and 2-amino-2-methylpropane-1,3-diol. 2-Amino-2-methylpropanol has proven to be an especially suitable neutralizing agent. Cosmetic agents preferred according to the invention therefore include 2-amino-2-methylpropanol. 2-Amino-2-methylpropanol is employed in the agents of the invention preferably in an amount that does not exceed the amount required for the neutralization of film-forming polymers a2). Preferably, the amounts, used in the agents of the invention, of 2-amino-2-methylpropanol are 80 to 100%, particularly preferably 90 to 100%, and in particular 95 to 100% of the amount required for the complete neutralization of film-forming polymers a2). In a preferred embodiment, the weight proportion of 2-amino-2-methylpropanol in terms of the total weight of the cosmetic preparation is 0.1 to 4.0% by weight, preferably 0.2 to 3.0% by weight, and especially 0.4 to 2.0% by weight.

The cosmetic agents of the invention include 40 to 95% by weight of ethanol as the third essential component, wherein it is preferred in regard to the production and application of the cosmetic agents that the weight proportion of ethanol a3) in terms of the total weight of cosmetic preparation a) is 50 to 95% by weight, preferably 70 to 95% by weight, and in particular 80 to 95% by weight. The weight proportion of water in terms of the total weight of cosmetic preparation a) in contrast is preferably less than 10% by weight, preferably less than 7% by weight, and in particular less than 5% by weight.

Agents of the invention are present in the form of flowable suspensions.

Apart from above-described ingredients, the cosmetic agents of the invention can include further active substances, auxiliary substances, and care substances.

A first group of preferred other active substances is formed by the poly-(C₂-C₃) alkylene glycol-modified silicones a4). Particularly preferred are poly-(C₂-C₃) alkylene glycol-modified silicones from the group of alkoxylated dimethicones, in particular
the ethoxylated dimethicones with the INCI name PEG-x Dimethicone with x=2 to 20, preferably 3 to 17, and in particular 11 or 12;
the ethoxylated dimethicones with the INCI name Bis-PEG-y Dimethicone with x=3 to 25, preferably 4 to 20;
the ethoxylated/propoxylated dimethicones with the INCI name PEG/PPG a/b Dimethicone, where a and b independently of one another stand for numbers from 2 to 30, preferably from 12 to 24, and in particular from 14 to 20;
the ethoxylated/propoxylated dimethicones with the INCI name bis-PEG/PPG-c/d Dimethicone, where c and d independently of one another stand for numbers from 10 to 25, preferably from 14 to 20, and in particular from 14 to 16;
the ethoxylated/propoxylated dimethicones with the INCI name Bis-PEG/PPG-e/f PEG/PPG g/h Dimethicone, where e, f, g, and h independently of one another stand for numbers from 10 to 20, preferably 14 to 18, and in particular 16.

The use of ethoxylated dimethicones with the INCI name PEG-12 Dimethicone is particularly preferred. The weight proportion of the poly-(C₂-C₃) alkylene glycol-modified silicones a4), preferably the ethoxylated dimethicones, in particular PEG-12 dimethicone, in terms of the total weight of cosmetic preparation a) is preferably 0.0001 to 1.0% by weight, preferably 0.0002 to 0.8% by weight, and in particular 0.0005 to 0.5% by weight.

The ester oils a5) form another group of preferred active substances. Ester oils in this case are understood to be the esters of C₆-C₃₀ fatty acids with C₂-C₃₀ fatty alcohols. The monoesters of the fatty acids with alcohols having 2 to 24 C atoms are preferred. Isopropyl myristate (Rilanit® IPM), isononanoic acid C₁₆₋₁₈ alkyl ester (Cetiol® SN), 2-ethylhexyl palmitate (Cegesoft® 24), stearic acid 2-ethylhexyl ester (Cetiol® 868), cetyl oleate, glycerol tricaprylate, coconut fatty alcohol caprinate/caprylate (Cetiol® LC), n-butyl stearate, oleyl erucate (Cetiol® J 600), isopropyl palmitate (Rilanit® IPP), oleyl oleate) (Cetiol®), lauric acid hexyl ester (Cetiol® A), di-n-butyl adipate (Cetiol® B), myristyl myristate (Cetiol® MM), cetearyl isononanoate (Cetiol® SN), and oleic acid decyl ester (Cetiol® V) are particularly preferred according to the invention. In preferred cosmetic agents of the invention, the weight proportion of ester oil a5) in terms of the total weight of cosmetic preparation a) is 0.01 to 1.0% by weight, preferably 0.02 to 0.8% by weight, and in particular 0.04 to 0.4% by weight.

Protein hydrolysates and/or derivatives thereof can be used as care substances. Protein hydrolysates are product mixtures obtained by acid-, base-, or enzyme-catalyzed degradation of proteins. The term ‘protein hydrolysates’ according to the invention is also understood to mean total hydrolysates, as well as individual amino acids and derivatives thereof, and mixtures of different amino acids. The molar weight of protein hydrolysates usable according to the invention is between 75 (the molar weight of glycine) and 200,000; the molar weight is preferably 75 to 50,000 daltons, and very particularly preferably 75 to 20,000 daltons.

A further group of care substances are vitamins, provitamins, vitamin precursors, and/or derivatives thereof. In this case, the vitamins, provitamins, and vitamin precursors of this kind that are usually assigned to the groups A, B, C, E, F, and H are preferred according to the invention.

Other care substances are glycerol, propylene glycol, panthenol, caffeine, nicotinamide, and sorbitol.

Plant extracts, but also mono- or oligosaccharides and/or lipids can be used as care substances.

The cosmetic agents of the invention are suitable in a particular way for application by means of a spray device, wherein the employed spray device can be a pump spray or an aerosol spray. Suitable cosmetic products accordingly comprise, apart from the above-described cosmetic agent a), furthermore a dispensing device with a spray valve b).

The use of aerosol sprays in which cosmetic agent a) is sprayed by a propellant gas under pressure is preferred. Suitable cosmetic products comprise, apart from the above-described cosmetic agent a) and a dispensing device with a spray valve b), furthermore a propellant c).

Propellants (propellant gases) suitable according to the invention are propane, propene, n-butane, isobutane, isobutene, n-pentane, pentene, isopentane, isopentene, methane, ethane, dimethyl ether, nitrogen, air, oxygen, laughing gas, 1,1,1,3-tetrafluoroethane, heptafluoro-n-propane, perfluoroethane, monochlorodifluoromethane, 1,1-difluoroethane, namely, both individually and also in combination. Hydrophilic propellant gases as well, such as, e.g., carbon dioxide, can be used advantageously in the context of the present invention, if the proportion of hydrophilic gases is selected as low and a lipophilic propellant gas (e.g., propane/butane) is present in excess. Propane, n-butane, mixtures of said propellant gases, and dimethyl ether are particularly preferred. It emerged that the use of dimethyl ether as the sole propellant gas brings with it advantages in production and use.

Preferably, the weight ratio of cosmetic preparation a) to propellant c) is 5:1 to 1:3, preferably 4:1 to 1:2, and in particular 2:1 to 1:1. Preferred cosmetic agents accordingly comprise
a cosmetic preparation a) including, based on the total weight of the cosmetic preparation,
   a1) 0.05 to 5.0% by weight of at least one hydrophobically modified metal oxide powder,
   a2) 0.1 to 10% by weight of at least one film-forming polymer,
   a3) 40 to 95% by weight of ethanol,
a propellant c), preferably dimethyl ether,
   where the weight ratio of cosmetic preparation a) to propellant c) is 5:1 to 1:3, preferably 4:1 to 1:2, and in particular 2:1 to 1:1.

In the case of the aerosol spray, the dispensing device with a spray valve usually also comprises a pressurized gas container. Vessels made of metal (aluminum, tin plate, tin), of protected or non-splintering plastic, or of glass that is coated on the outside with plastic may be used as compressed-gas containers; pressure and breaking strength, corrosion resistance, easy fillability, as well as aesthetic aspects, handling, printability, etc., play a role in their selection. Special protective interior coatings assure corrosion resistance to cosmetic agent a).

In a preferred embodiment of the invention, the valve has a valve cone covered with a coating or a polymeric plastic A, and a flexible element of this kind with a reset function, which after the operation ends resets the valve to the closed position (=neutral position of the valve). Corresponding cosmetic products in which the aerosol dispensing device comprises a valve, which has a valve cone and/or a flexible element with a reset function which is/are covered with a coating or a polymeric plastic A, are preferred according to the invention.

In another preferred embodiment of the invention, the valve has a flexible element with a reset function and/or a valve cone made of at least one plastic B, preferably an elastomeric plastic. Here as well, cosmetic products of the invention in which the valve has a flexible element with a reset function and/or a valve cone made of at least one plastic B, are preferred, preferred plastics B being elastomeric plastics. Especially preferred elastomeric plastics are selected from Buna, particularly Buna N, Buna 421, Buna 1602, and Buna KA 6712, neoprene, butyl, and chlorobutyl.

In another preferred embodiment of the invention, the flexible element with a reset function can be formed as a spiral spring or helical compression spring. In another preferred embodiment of the invention, the flexible element with the reset function can be formed integrally with the valve cone and have flexible legs.

The composition of some preferred cosmetic preparations can be obtained from the following tables (data are given in % by weight, based on the total weight of the cosmetic preparation, unless otherwise stated).

| | Formula 1 | Formula 2 | Formula 3 | Formula 4 | Formula 5 |
|---|---|---|---|---|---|
| Hydrophobically | 0.05 to 5.0 | 0.1 to 4.0 | 0.15 to 3.0 | 0.15 to 3.0 | 0.2 to 2.0 |
| modified metal oxide | | | | | |
| powder a) | | | | | |
| Film-forming polymer b) | 0.1 to 10 | 0.2 to 8.0 | 0.4 to 6.0 | 0.5 to 5.0 | 0.5 to 5.0 |
| Ethanol | 40 to 95 | 40 to 95 | 50 to 95 | 70 to 95 | 80 to 95 |
| Optional additives | To 100 | To 100 | To 100 | To 100 | To 100 |
| | Formula 6 | Formula 7 | Formula 8 | Formula 9 | Formula 10 |
| Pyrogenic silicon dioxide | 0.05 to 5.0 | 0.1 to 4.0 | 0.15 to 3.0 | 0.15 to 3.0 | 0.2 to 2.0 |
| hydrophobized by | | | | | |
| silanization a) | | | | | |
| Film-forming polymer b) | 0.1 to 10 | 0.2 to 8.0 | 0.4 to 6.0 | 0.5 to 5.0 | 0.5 to 5.0 |
| Ethanol | 40 to 95 | 40 to 95 | 50 to 95 | 70 to 95 | 80 to 95 |
| Optional additives | To 100 | To 100 | To 100 | To 100 | To 100 |
| | Formula 11 | Formula 12 | Formula 13 | Formula 14 | Formula 15 |
| Hydrophobically | 0.05 to 5.0 | 0.1 to 4.0 | 0.15 to 3.0 | 0.15 to 3.0 | 0.2 to 2.0 |
| modified metal oxide | | | | | |
| powder a) | | | | | |
| Octylacrylamide/ | 0.1 to 10 | 0.2 to 8.0 | 0.4 to 6.0 | 0.5 to 5.0 | 0.5 to 5.0 |
| Acrylates/ | | | | | |
| Butylaminoethyl | | | | | |
| Methacrylate Copolymer b) | | | | | |
| Ethanol | 40 to 95 | 40 to 95 | 50 to 95 | 70 to 95 | 80 to 95 |
| Optional additives | To 100 | To 100 | To 100 | To 100 | To 100 |
| | Formula 16 | Formula 17 | Formula 18 | Formula 19 | Formula 20 |
| Silica Dimethyl Silylate | 0.05 to 5.0 | 0.1 to 4.0 | 0.15 to 3.0 | 0.15 to 3.0 | 0.2 to 2.0 |
| Film-forming polymer b) | 0.1 to 10 | 0.2 to 8.0 | 0.4 to 6.0 | 0.5 to 5.0 | 0.5 to 5.0 |
| Ethanol | 40 to 95 | 40 to 95 | 50 to 95 | 70 to 95 | 80 to 95 |
| Optional additives | To 100 | To 100 | To 100 | To 100 | To 100 |
| | Formula 21 | Formula 22 | Formula 23 | Formula 24 | Formula 25 |
| Silica Dimethyl Silylate | 0.05 to 5.0 | 0.1 to 4.0 | 0.15 to 3.0 | 0.15 to 3.0 | 0.2 to 2.0 |
| Octylacrylamide/ | 0.1 to 10 | 0.2 to 8.0 | 0.4 to 6.0 | 0.5 to 5.0 | 0.5 to 5.0 |
| Acrylates/ | | | | | |
| Butylaminoethyl | | | | | |
| Methacrylate Copolymer b) | | | | | |
| Ethanol | 40 to 95 | 40 to 95 | 50 to 95 | 70 to 95 | 80 to 95 |
| Optional additives | To 100 | To 100 | To 100 | To 100 | To 100 |
| | Formula 26 | Formula 27 | Formula 28 | Formula 29 | Formula 30 |
| Hydrophobically | 0.05 to 5.0 | 0.1 to 4.0 | 0.15 to 3.0 | 0.15 to 3.0 | 0.2 to 2.0 |
| modified metal oxide | | | | | |
| powder a) | | | | | |
| Film-forming polymer b) | 0.1 to 10 | 0.2 to 8.0 | 0.4 to 6.0 | 0.5 to 5.0 | 0.5 to 5.0 |
| Ester oil | 0.01 to 10 | 0.02 to 0.8 | 0.02 to 0.8 | 0.94 to 0.4 | 0.04 to 0.4 |
| Ethanol | 40 to 95 | 40 to 95 | 50 to 95 | 70 to 95 | 80 to 95 |
| Optional additives | To 100 | To 100 | To 100 | To 100 | To 100 |
| | Formula 31 | Formula 32 | Formula 33 | Formula 34 | Formula 35 |
| Pyrogenic silicon dioxide | 0.05 to 5.0 | 0.1 to 4.0 | 0.15 to 3.0 | 0.15 to 3.0 | 0.2 to 2.0 |
| hydrophobized by | | | | | |
| silanization a) | | | | | |
| Film-forming polymer b) | 0.1 to 10 | 0.2 to 8.0 | 0.4 to 6.0 | 0.5 to 5.0 | 0.5 to 5.0 |
| Ester oil | 0.01 to 10 | 0.02 to 0.8 | 0.02 to 0.8 | 0.94 to 0.4 | 0.04 to 0.4 |
| Ethanol | 40 to 95 | 40 to 95 | 50 to 95 | 70 to 95 | 80 to 95 |
| Optional additives | To 100 | To 100 | To 100 | To 100 | To 100 |
| | Formula 36 | Formula 37 | Formula 38 | Formula 39 | Formula 40 |
| Hydrophobically | 0.05 to 5.0 | 0.1 to 4.0 | 0.15 to 3.0 | 0.15 to 3.0 | 0.2 to 2.0 |
| modified metal oxide | | | | | |
| powder a) | | | | | |
| Octylacrylamide/ | 0.1 to 10 | 0.2 to 8.0 | 0.4 to 6.0 | 0.5 to 5.0 | 0.5 to 5.0 |
| Acrylates/ | | | | | |
| Butylaminoethyl | | | | | |
| Methacrylate Copolymer b) | | | | | |
| Ester oil | 0.01 to 10 | 0.02 to 0.8 | 0.02 to 0.8 | 0.94 to 0.4 | 0.04 to 0.4 |
| Ethanol | 40 to 95 | 40 to 95 | 50 to 95 | 70 to 95 | 80 to 95 |
| Optional additives | To 100 | To 100 | To 100 | To 100 | To 100 |
| | Formula 41 | Formula 42 | Formula 43 | Formula 44 | Formula 45 |
| Silica Dimethyl Silylate | 0.05 to 5.0 | 0.1 to 4.0 | 0.15 to 3.0 | 0.15 to 3.0 | 0.2 to 2.0 |
| Film-forming polymer b) | 0.1 to 10 | 0.2 to 8.0 | 0.4 to 6.0 | 0.5 to 5.0 | 0.5 to 5.0 |
| Ester oil | 0.01 to 10 | 0.02 to 0.8 | 0.02 to 0.8 | 0.94 to 0.4 | 0.04 to 0.4 |
| Ethanol | 40 to 95 | 40 to 95 | 50 to 95 | 70 to 95 | 80 to 95 |
| Optional additives | To 100 | To 100 | To 100 | To 100 | To 100 |
| | Formula 46 | Formula 47 | Formula 48 | Formula 49 | Formula 50 |
| Silica Dimethyl Silylate | 0.05 to 5.0 | 0.1 to 4.0 | 0.15 to 3.0 | 0.15 to 3.0 | 0.2 to 2.0 |
| Octylacrylamide/ | 0.1 to 10 | 0.2 to 8.0 | 0.4 to 6.0 | 0.5 to 5.0 | 0.5 to 5.0 |
| Acrylates/ | | | | | |
| Butylaminoethyl | | | | | |
| Methacrylate Copolymer b) | | | | | |
| Ester oil | 0.01 to 10 | 0.02 to 0.8 | 0.02 to 0.8 | 0.94 to 0.4 | 0.04 to 0.4 |
| Ethanol | 40 to 95 | 40 to 95 | 50 to 95 | 70 to 95 | 80 to 95 |
| Optional additives | To 100 | To 100 | To 100 | To 100 | To 100 |
| | Formula 51 | Formula 52 | Formula 53 | Formula 54 | Formula 55 |
| Silica Dimethyl Silylate | 0.05 to 5.0 | 0.1 to 4.0 | 0.15 to 3.0 | 0.15 to 3.0 | 0.2 to 2.0 |
| Octylacrylamide/ | 0.1 to 10 | 0.2 to 8.0 | 0.4 to 6.0 | 0.5 to 5.0 | 0.5 to 5.0 |
| Acrylates/ | | | | | |
| Butylaminoethyl | | | | | |
| Methacrylate Copolymer b) | | | | | |
| Isopropyl myristate | 0.01 to 10 | 0.02 to 0.8 | 0.02 to 0.8 | 0.94 to 0.4 | 0.04 to 0.4 |
| Ethanol | 40 to 95 | 40 to 95 | 50 to 95 | 70 to 95 | 80 to 95 |
| Optional additives | To 100 | To 100 | To 100 | To 100 | To 100 |

The further composition of some preferred cosmetic agents, which comprise apart from cosmetic preparation a) further a propellant c), can be obtained from the following tables. In this table, the left column (“Formula x”) in each case refers to one of the exemplary cosmetic preparations listed in the tables disclosed further above. The other columns two to seven (“propellant”) in each case indicate the amount of propellant added to the particular cosmetic preparation. These quantities given in “% by weight” refer to the total weight of cosmetic preparation a) of the particular “Formula x” without propellant.

The cosmetic agents according to row 2, column 6 in the following table in other words are mixtures of the propellant-free cosmetic preparation a) according to formula 1 with dimethyl ether in a weight ratio of 2:1 to 1:1.

In row 4, column 5 of the following table, in contrast, a 3:2 mixture of the propellant-free cosmetic preparation a) according to formula 3 with a propane/butane mixture is described.

| | Propellant [% by weight] | | | | | |
|---|---|---|---|---|---|---|
| Formula 1 | 50 to 100 | 66.6 | 50 to 100 P/B* | 66.6 P/B | 50 to 100 | 66.6 DME |
| | | | | | DME** | |
| Formula 2 | 50 to 100 | 66.6 | 50 to 100 P/B* | 66.6 P/B | 50 to 100 | 66.6 DME |
| | | | | | DME** | |
| Formula 3 | 50 to 100 | 66.6 | 50 to 100 P/B* | 66.6 P/B | 50 to 100 | 66.6 DME |
| | | | | | DME** | |
| Formula 4 | 50 to 100 | 66.6 | 50 to 100 P/B* | 66.6 P/B | 50 to 100 | 66.6 DME |
| | | | | | DME** | |
| Formula 5 | 50 to 100 | 66.6 | 50 to 100 P/B* | 66.6 P/B | 50 to 100 | 66.6 DME |
| | | | | | DME** | |
| Formula 6 | 50 to 100 | 66.6 | 50 to 100 P/B* | 66.6 P/B | 50 to 100 | 66.6 DME |
| | | | | | DME** | |
| Formula 7 | 50 to 100 | 66.6 | 50 to 100 P/B* | 66.6 P/B | 50 to 100 | 66.6 DME |
| | | | | | DME** | |
| Formula 8 | 50 to 100 | 66.6 | 50 to 100 P/B* | 66.6 P/B | 50 to 100 | 66.6 DME |
| | | | | | DME** | |
| Formula 9 | 50 to 100 | 66.6 | 50 to 100 P/B* | 66.6 P/B | 50 to 100 | 66.6 DME |
| | | | | | DME** | |
| Formula 10 | 50 to 100 | 66.6 | 50 to 100 P/B* | 66.6 P/B | 50 to 100 | 66.6 DME |
| | | | | | DME** | |
| Formula 11 | 50 to 100 | 66.6 | 50 to 100 P/B* | 66.6 P/B | 50 to 100 | 66.6 DME |
| | | | | | DME** | |
| Formula 12 | 50 to 100 | 66.6 | 50 to 100 P/B* | 66.6 P/B | 50 to 100 | 66.6 DME |
| | | | | | DME** | |
| Formula 13 | 50 to 100 | 66.6 | 50 to 100 P/B* | 66.6 P/B | 50 to 100 | 66.6 DME |
| | | | | | DME** | |
| Formula 14 | 50 to 100 | 66.6 | 50 to 100 P/B* | 66.6 P/B | 50 to 100 | 66.6 DME |
| | | | | | DME** | |
| Formula 15 | 50 to 100 | 66.6 | 50 to 100 P/B* | 66.6 P/B | 50 to 100 | 66.6 DME |
| | | | | | DME** | |
| Formula 16 | 50 to 100 | 66.6 | 50 to 100 P/B* | 66.6 P/B | 50 to 100 | 66.6 DME |
| | | | | | DME** | |
| Formula 17 | 50 to 100 | 66.6 | 50 to 100 P/B* | 66.6 P/B | 50 to 100 | 66.6 DME |
| | | | | | DME** | |
| Formula 18 | 50 to 100 | 66.6 | 50 to 100 P/B* | 66.6 P/B | 50 to 100 | 66.6 DME |
| | | | | | DME** | |
| Formula 19 | 50 to 100 | 66.6 | 50 to 100 P/B* | 66.6 P/B | 50 to 100 | 66.6 DME |
| | | | | | DME** | |
| Formula 20 | 50 to 100 | 66.6 | 50 to 100 P/B* | 66.6 P/B | 50 to 100 | 66.6 DME |
| | | | | | DME** | |
| Formula 21 | 50 to 100 | 66.6 | 50 to 100 P/B* | 66.6 P/B | 50 to 100 | 66.6 DME |
| | | | | | DME** | |
| Formula 22 | 50 to 100 | 66.6 | 50 to 100 P/B* | 66.6 P/B | 50 to 100 | 66.6 DME |
| | | | | | DME** | |
| Formula 23 | 50 to 100 | 66.6 | 50 to 100 P/B* | 66.6 P/B | 50 to 100 | 66.6 DME |
| | | | | | DME** | |
| Formula 24 | 50 to 100 | 66.6 | 50 to 100 P/B* | 66.6 P/B | 50 to 100 | 66.6 DME |
| | | | | | DME** | |
| Formula 25 | 50 to 100 | 66.6 | 50 to 100 P/B* | 66.6 P/B | 50 to 100 | 66.6 DME |
| | | | | | DME** | |
| Formula 26 | 50 to 100 | 66.6 | 50 to 100 P/B* | 66.6 P/B | 50 to 100 | 66.6 DME |
| | | | | | DME** | |
| Formula 27 | 50 to 100 | 66.6 | 50 to 100 P/B* | 66.6 P/B | 50 to 100 | 66.6 DME |
| | | | | | DME** | |
| Formula 28 | 50 to 100 | 66.6 | 50 to 100 P/B | 66.6 P/B | 50 to 100 | 66.6 DME |
| | | | | | DME** | |
| Formula 29 | 50 to 100 | 66.6 | 50 to 100 P/B* | 66.6 P/B | 50 to 100 | 66.6 DME |
| | | | | | DME** | |
| Formula 30 | 50 to 100 | 66.6 | 50 to 100 P/B* | 66.6 P/B | 50 to 100 | 66.6 DME |
| | | | | | DME** | |
| Formula 31 | 50 to 100 | 66.6 | 50 to 100 P/B* | 66.6 P/B | 50 to 100 | 66.6 DME |
| | | | | | DME** | |
| Formula 32 | 50 to 100 | 66.6 | 50 to 100 P/B* | 66.6 P/B | 50 to 100 | 66.6 DME |
| | | | | | DME** | |
| Formula 33 | 50 to 100 | 66.6 | 50 to 100 P/B* | 66.6 P/B | 50 to 100 | 66.6 DME |
| | | | | | DME** | |
| Formula 34 | 50 to 100 | 66.6 | 50 to 100 P/B* | 66.6 P/B | 50 to 100 | 66.6 DME |
| | | | | | DME** | |
| Formula 35 | 50 to 100 | 66.6 | 50 to 100 P/B* | 66.6 P/B | 50 to 100 | 66.6 DME |
| | | | | | DME** | |
| Formula 36 | 50 to 100 | 66.6 | 50 to 100 P/B* | 66.6 P/B | 50 to 100 | 66.6 DME |
| | | | | | DME** | |
| Formula 37 | 50 to 100 | 66.6 | 50 to 100 P/B* | 66.6 P/B | 50 to 100 | 66.6 DME |
| | | | | | DME** | |
| Formula 38 | 50 to 100 | 66.6 | 50 to 100 P/B* | 66.6 P/B | 50 to 100 | 66.6 DME |
| | | | | | DME** | |
| Formula 39 | 50 to 100 | 66.6 | 50 to 100 P/B* | 66.6 P/B | 50 to 100 | 66.6 DME |
| | | | | | DME** | |
| Formula 40 | 50 to 100 | 66.6 | 50 to 100 P/B* | 66.6 P/B | 50 to 100 | 66.6 DME |
| | | | | | DME** | |
| Formula 41 | 50 to 100 | 66.6 | 50 to 100 P/B* | 66.6 P/B | 50 to 100 | 66.6 DME |
| | | | | | DME** | |
| Formula 42 | 50 to 100 | 66.6 | 50 to 100 P/B* | 66.6 P/B | 50 to 100 | 66.6 DME |
| | | | | | DME** | |
| Formula 43 | 50 to 100 | 66.6 | 50 to 100 P/B* | 66.6 P/B | 50 to 100 | 66.6 DME |
| | | | | | DME** | |
| Formula 44 | 50 to 100 | 66.6 | 50 to 100 P/B* | 66.6 P/B | 50 to 100 | 66.6 DME |
| | | | | | DME** | |
| Formula 45 | 50 to 100 | 66.6 | 50 to 100 P/B* | 66.6 P/B | 50 to 100 | 66.6 DME |
| | | | | | DME** | |
| Formula 46 | 50 to 100 | 66.6 | 50 to 100 P/B* | 66.6 P/B | 50 to 100 | 66.6 DME |
| | | | | | DME** | |
| Formula 47 | 50 to 100 | 66.6 | 50 to 100 P/B* | 66.6 P/B | 50 to 100 | 66.6 DME |
| | | | | | DME** | |
| Formula 48 | 50 to 100 | 66.6 | 50 to 100 P/B* | 66.6 P/B | 50 to 100 | 66.6 DME |
| | | | | | DME** | |
| Formula 49 | 50 to 100 | 66.6 | 50 to 100 P/B* | 66.6 P/B | 50 to 100 | 66.6 DME |
| | | | | | DME** | |
| Formula 50 | 50 to 100 | 66.6 | 50 to 100 P/B* | 66.6 P/B | 50 to 100 | 66.6 DME |
| | | | | | DME** | |
| Formula 51 | 50 to 100 | 66.6 | 50 to 100 P/B* | 66.6 P/B | 50 to 100 | 66.6 DME |
| | | | | | DME** | |
| Formula 52 | 50 to 100 | 66.6 | 50 to 100 P/B* | 66.6 P/B | 50 to 100 | 66.6 DME |
| | | | | | DME** | |
| Formula 53 | 50 to 100 | 66.6 | 50 to 100 P/B* | 66.6 P/B | 50 to 100 | 66.6 DME |
| | | | | | DME** | |
| Formula 54 | 50 to 100 | 66.6 | 50 to 100 P/B* | 66.6 P/B | 50 to 100 | 66.6 DME |
| | | | | | DME** | |
| Formula 55 | 50 to 100 | 66.6 | 50 to 100 P/B* | 66.6 P/B | 50 to 100 | 66.6 DME |
| | | | | | DME** | |

“P/B” corresponds to a propane/butane mixture
“DME” corresponds to dimethyl ether

As initially stated, the previously described cosmetic agents are characterized by particular hair-cosmetic properties, in particular advantageous properties during temporary hair shaping. A second subject of the present application therefore is the use of an agent of the invention for the temporary shaping of keratin-containing fibers, in particular human hair.

A third subject of the present application is a method for the temporary shaping of keratin-containing fibers, in particular human hair, in which the keratinic fibers are acted upon by a cosmetic agent of the invention and are fixed temporarily in their shape.

The agents, uses, and methods of the invention and some of their preferred embodiments are characterized by the following points:

A cosmetic agent for the temporary shaping of keratinic fibers, comprising

a) a cosmetic preparation including, based on the total weight of the cosmetic preparation,
a1) 0.05 to 5.0% by weight of at least one hydrophobically modified metal oxide powder,
a2) 0.1 to 10% by weight of at least one film-forming polymer,
a3) 40 to 95% by weight of ethanol.

The cosmetic agent according to point 1, characterized in that the weight proportion of the hydrophobically modified metal oxide powder a1) in terms of the total weight of cosmetic preparation a) is 0.1 to 4.0% by weight, preferably 0.15 to 3.0% by weight, and in particular 0.2 to 2.0% by weight.

The cosmetic agent according to one of the preceding points, characterized in that the hydrophobically modified metal oxide powder a) is selected from the group of hydrophobized silicates obtained by silanization of pyrogenic silicon dioxide.

The cosmetic agent according to one of the preceding points, characterized in that the hydrophobically modified metal oxide powder a) is selected from the compounds with the INCI names: Silica Dimethyl Silylate (e.g., Aerosil R792, Aerosil R794), Silica Dimethicone Silylate (e.g., Aerosil R202), and Silica Silylate (e.g., Aerosil R805, Aerosil R812, Aerosil R816), preferably from the compounds with the INCI name Silica Dimethyl Silylate.

The cosmetic agent according to one of the preceding points, characterized in that the weight proportion of film-forming polymer a2) in terms of the total weight of cosmetic preparation a) is 0.2 to 8.0% by weight, preferably 0.4 to 6.0% by weight, and in particular 0.5 to 5.0% by weight.

The cosmetic agent according to one of the preceding points, characterized in that film-forming polymer a2) is a copolymer of the monomers
i) N-tert-octylacrylamide,
ii) acrylic acid,
iii) tert-butylaminoethyl methacrylic acid,
iv) and optionally other monomers.

The cosmetic agents according to one of the preceding points, characterized in that film-forming polymer a2) is a copolymer from the group of polyvinylpyrrolidone and vinylpyrrolidone/vinyl acetate copolymers, preferably the polyvinylpyrrolidones.

The cosmetic agent according to one of the preceding points, characterized in that film-forming polymer a2) is a copolymer of the monomers
iv) acrylic acid,
v) ethyl acrylate,
vi) tert-butylacrylamide.

The cosmetic agent according to one of the preceding points, characterized in that the weight proportion of ethanol a3) in terms of the total weight of cosmetic preparation a) is 50 to 95% by weight, preferably 70 to 95% by weight, and in particular 80 to 95% by weight.

The cosmetic agent according to one of the preceding points, characterized in that the weight proportion of water in terms of the total weight of cosmetic preparation a) is less than 10% by weight, preferably less than 7% by weight, and in particular less than 5% by weight.

A cosmetic product comprising
a) a cosmetic agent according to one of points 1 to 10,
b) a dispensing device with a spray valve.

A cosmetic product comprising
a) a cosmetic agent according to one of points 1 to 10,
b) a dispensing device with a spray valve,
c) a propellant.

The cosmetic products according to point 12, characterized in that the weight ratio of cosmetic preparation a) to propellant c) is 5:1 to 1:3, preferably 4:1 to 1:2, and in particular 2:1 to 1:1.

Use of an agent or a product according to one of points 1 to 13 for the temporary shaping of keratin-containing fibers, in particular human hair.

A method for the temporary shaping of keratin-containing fibers, in particular human hair, in which the keratinic fibers are acted upon by a cosmetic agent according to one of points 1 to 10 and are temporarily fixed in their shape.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. A cosmetic agent for the temporary shaping of keratinic fibers, comprising
a) a cosmetic preparation including, based on the total weight of the cosmetic preparation,
a1) 0.05 to 5.0% by weight of at least one hydrophobically modified metal oxide powder,
a2) 0.1 to 10% by weight of at least one film-forming polymer,
a3) 40 to 95% by weight of ethanol.

2. The cosmetic agent according to claim 1, wherein the weight proportion of the hydrophobically modified metal oxide powder a1) in terms of the total weight of cosmetic preparation a) is 0.1 to 4.0% by weight

3. The cosmetic agent according to claim 1, wherein the weight proportion of the hydrophobically modified metal oxide powder a) in terms of the total weight of cosmetic preparation a) is 0.15 to 3.0% by weight.

4. The cosmetic agent according to claim 1, wherein the weight proportion of the hydrophobically modified metal oxide powder a) in terms of the total weight of cosmetic preparation a) is 0.2 to 2.0% by weight.

5. The cosmetic agent according to claim 1, wherein the hydrophobically modified metal oxide powder a) is selected from the group consisting of hydrophobized silicates obtained by silanization of pyrogenic silicon dioxide.

6. The cosmetic agent according to claim 1, wherein the weight proportion of film-forming polymer a2) in terms of the total weight of cosmetic preparation a) is 0.2 to 8.0% by weight.

7. The cosmetic agent according to claim 1, wherein the weight proportion of film-forming polymer a2) in terms of the total weight of cosmetic preparation a) 0.4 to 6.0% by weight.

8. The cosmetic agent according to claim 1, wherein the weight proportion of film-forming polymer a2) in terms of the total weight of cosmetic preparation a) 0.5 to 5.0% by weight.

9. The cosmetic agent according to claim 1, wherein the film-forming polymer a2) is a copolymer of the monomers
i) N-tert-octylacrylamide,
ii) acrylic acid,
iii) tert-butylaminoethyl methacrylic acid,
iv) and optionally other monomers.

10. The cosmetic agent according to claim 1, wherein the weight proportion of ethanol a3) in terms of the total weight of cosmetic preparation a) is 50 to 95% by weight.

11. The cosmetic agent according to claim 1, wherein the weight proportion of ethanol a3) in terms of the total weight of cosmetic preparation a) is 70 to 95% by weight.

12. The cosmetic agent according to claim 1, wherein the weight proportion of ethanol a3) in terms of the total weight of cosmetic preparation a) is 80 to 95% by weight.

13. A cosmetic product comprising
a) a cosmetic agent according to claim 1,
b) a dispensing device with a spray valve.

14. A cosmetic product comprising
a) a cosmetic agent according to claim 1;
b) a dispensing device with a spray valve,
c) a propellant.

15. A method for the temporary shaping of keratin-containing fibers, comprising:
applying a cosmetic agent of claim 1 to keratinic fibers; and
shaping the keratinic fibers.
